(19) United States
(12) Patent Application Publication
(10) Pub. No.: US 2014/0052042 A1
(43) Pub. Date: Feb. 20, 2014

(54) MEDICAL DEVICE

(71) Applicants: Philip Russo, Surrey (GB); Debra Russo, Surrey (GB)

(72) Inventors: Philip Russo, Surrey (GB); Debra Russo, Surrey (GB)

(21) Appl. No.: 13/969,685

(22) Filed: Aug. 19, 2013

(30) Foreign Application Priority Data
Aug. 17, 2012 (GB) 1214741.9
Jan. 31, 2013 (GB) 1301687.8

(51) Int. Cl. A61F 5/32

(52) U.S. Cl. CPC A61F 5/32
USPC 602/53

(57) ABSTRACT

A medical device for covered wound relief, comprising: an annular pressure member for separating a skin fold covering or overlapping a wound, and wherein the pressure member has at least one aperture extending therethrough for aeration of the wound.

This invention relates generally to medical devices that provide relief for wounds such as blisters, sores and the like. The invention has particular, but not exclusive, application in areas where the skin forms folds or creases, such as the perineal or anal region where wounds such as haemorrhoids, anal fissures and fistulas are often formed.

Anal and rectal conditions such as haemorrhoids, fistulas, setons, anal fissures and the like can be particularly painful for the sufferer. Devices for relief are known, in general employing either belts or straps to keep the device in place which can be both uncomfortable and unhygienic if not regularly cleaned and sterilised.

Other devices have been proposed which are secured in place using invasive plugs which can be painful when inserted in an already inflamed and painful anus. Furthermore, applying such devices in an area that is already poorly ventilated, itchy and irritated by bodily fluids such as perspiration or weeping from wounds may actually increase discomfort and itching.

Other wounds in areas where the skin forms creases or folds are poorly, if at all, ventilated since the wounds may be substantially covered. Such areas comprise the anal, groin, abdominal, chest and armpit regions for example. The wounds therein can be difficult to provide aeration and relief thereto.

In general, persons who lead sedentary lifestyles or occupations are often susceptible to such wounds. For instance, pilots, lorry, truck or taxi drivers, office workers and the like are known to suffer incidence of haemorrhoids and the like.

There is hence a desire to improve on the above by providing a device that is relatively lightweight, comfortable, retains position once placed in-situ and aids ventilation in the affected wound area.

According to a first aspect of the present invention, there is provided a medical device for covered wound relief. The device comprises a pressure member for separating a skin fold covering or overlapping a wound, and the pressure member has at least one aperture extending therethrough, and wherein the pressure member comprises an internal channel extending around the aperture.

Owing to the invention, the device is easily applied to provide pressure to separate skin folds, such as the buttocks, in the vicinity of an affected wound area, whilst simultaneously providing aeration to the wound area. The device may therefore be left in-situ whilst the patient is sleeping or in a resting position, thereby providing night comfort.

In a preferred embodiment, the pressure member is an annular pressure member.

The channel may be an enclosed internal channel or an embedded channel. The channel may comprise a sealed tube, a re-sealable tube or a cavity.

The channel may comprise more than one cavity dispersed throughout the pressure member.

In some preferred embodiments, the channel may comprise a medium. The medium may be a fluid and/or a gel. The medium may be capable of being cooled and/or heated. The medium may comprise cold pack fluid or gel. The cold pack fluid or gel may be capable of holding heat or capable of cooling.

The cold pack fluid or gel may comprise water, a gelling agent and a salt. The gelling agent may be selected from a group of gelling agents including silica gel, including vinyl coated silica gel, cellulose, including hydroxyethyl cellulose (cellusize), salts of carboxymethyl cellulose, including sodium carboxymethyl cellulose. The salt may be selected from a group of salts including ammonium salts, including ammonium nitrate or ammonium chloride, urea salts, potassium nitrate, calcium chloride, magnesium sulphate, sodium acetate.

The cold pack fluid or gel may further comprise a freezing point depressant. The freezing point depressant may be propylene glycol.

In another embodiment, the device is provided with an internal channel which may comprise a first section, a second section, and a breakable membrane separating the first section from the second section, the membrane being breakable by flexing or compressing the device. The internal channel may be an embedded channel which is divided into two sections separated by a breakable membrane.

The membrane is broken when the device is flexed or compressed prior to placement, thereby generating a cooling and/or heating effect in addition to aeration.

The first section may comprise a first medium and the second section may comprise a second medium. The medium may be compositions. They may be the same or different compositions. The compositions may comprise chemical compounds. For example, the first medium may comprise water, and the second medium may comprise compounds selected from the group of ammonium nitrate or ammonium chloride, urea salts, potassium nitrate, calcium chloride, magnesium sulphate, sodium acetate.

In one embodiment, when the first and second compositions are mixed, an endothermic reaction may occur. Alternatively, in another embodiment, when the first and second compositions are mixed, an exothermic reaction may occur.

The device may also be used to help prevent or provide relief from sores between skin folds in the abdominal area, which can be a particular problem for bed-ridden, or overweight or obese patients.

In an embodiment the device is substantially annular and preferably oval in shape. Alternatively, the pressure member may be rhomboidal in shape. The aperture may be located centrally or asymmetrically.

The device is preferably formed from resilient, flexible material such as natural or synthetic rubber, plastic, silicone, foam or a mixture thereof. The foam may be low, medium or high density foam.

Those skilled in the art will appreciate that modern extruding techniques and materials such as those used in the production of polyvinylchloride (PVC), nitrile PVC, and PVC-silicone may be utilised to form the device in a relatively inexpensive and mass production compatible fashion.

The pressure member may comprise an inner surface defining the at least one aperture and an outer surface defining the periphery of the pressure member, and wherein the outer surface comprises an upper surface and a lower surface.

In another embodiment the device has tapered sidewalls between the upper and lower surfaces of the pressure member, providing a “wedge” shape in cross-section to enable easier placement and conformity with skin folds.

In another embodiment, the pressure member comprises an inner surface defining the at least one aperture and an outer surface defining an outer periphery of the pressure member.

The outer surface may include first and second surface sections which extend radially outwardly in spaced relation to the inner surface.

One surface section may have an arcuate form. In an alternative embodiment, the surface sections both have an arcuate form.

The surface sections may be convex surfaces. In an alternative embodiment, the surface sections may be concave surfaces.

Each arcuate surface may be symmetric. Alternatively, each arcuate surface is asymmetric.

In one embodiment, the sidewall may taper inwards from the upper to the lower surface at an angle in the range 5 to 30 degrees with respect to a vertical axis extending through the at least one aperture. In other embodiments, the side wall may taper inward in the range 5 to 45 degrees, and preferably around 20 degrees. The angle may be 5, 10, 15, 20, 25, 30, 35, 40, 45 degrees.

The surface sections may comprise a first major element which extends substantially in a first plane, and a second, minor element which extends substantially in a second plane which encloses an obtuse angle with the first plane.

The outer surface may comprise an outer sidewall which extends between the radially outer periphery of the first and second surface sections.

The outer sidewall is inclined relative to an axial direction extending between the first and second surface sections.

According to another aspect of the invention, there is provided a medical device for covered wound relief, comprising an annular pressure member for separating a skin fold covering or overlapping a wound, and wherein the pressure member has at least one aperture extending therethrough, and wherein the pressure member comprises an inner surface defining the at least one aperture and an outer surface defining the periphery of the pressure member, and wherein the outer surface includes first and second surface sections which extend radially outwardly in spaced relation to the inner surface, and wherein the outer surface comprises an outer sidewall which extends between the radially outer periphery of the first and second surface sections, and wherein the outer sidewall is inclined relative to an axial direction extending between the first and second surface sections.

According to another aspect of the invention, there is provided a medical device for covered wound relief, comprising an annular pressure member for separating a skin fold covering or overlapping a wound, and wherein the pressure member has at least one aperture extending therethrough, and wherein the pressure member comprises an inner surface defining the at least one aperture and an outer surface defining the periphery of the pressure member, and wherein the outer surface comprises an upper surface and a lower surface, a sidewall extending continuously from the lower surface to the upper surface, and wherein the sidewall tapers inwards from the upper surface to the lower surface.

In one embodiment, the sidewall may taper inwards from the upper to the lower surface at an angle in the range 5 to 30 degrees with respect to a vertical axis extending through the at least one aperture. In other embodiments, the side wall may taper inward in the range 5 to 45 degrees, and preferably around 20 degrees. The angle may be 5, 10, 15, 20, 25, 30, 35, 40, 45 degrees.

One surface section may have an arcuate form. In an alternative embodiment, the surface sections both have an arcuate form.

In another embodiment the device has a convex curved upper surface. In another embodiment, the device has a convex curved lower surface. The surface sections may be convex surfaces. In an alternative embodiment, the surface sections may be concave surfaces.

In one embodiment, the curved surface is symmetric. In an alternative embodiment, the curved surface is asymmetric. Each arcuate surface may be symmetric. Alternatively, each arcuate surface is asymmetric.

In an embodiment, the device outer surface is textured. This is to enhance grip once the device is placed in-situ.

In yet a further embodiment, the device comprises more than one aperture. The device may comprise 1, 2, 3, 4, 5, 6, 7, 8, 9, 10 apertures. The apertures may be of different sizes.

In another embodiment, the device has an outer surface portion that is more absorbent relative to the remaining portion of the device. The outer portion may be provided with or impregnated with astringent, antiseptic, antibiotic or anti-bacterial fluids or compounds.

In yet a further embodiment, the device has an outer surface portion that is made of absorbent or hydrophilic foam that can be loaded or impregnated with antiseptic, antibiotic and/or anti-bacterial compositions, which may be fluids, agents or chemicals. The device can therefore also absorb weeping fluids and perspiration to aid comfort.

In another embodiment, the outer surface portion is impregnated with antibacterial agents or sterilising compounds during manufacture.

In another embodiment, the device comprises surgical tissue that is adhered to the contours of the pressure member.

In yet another embodiment, the pressure member of the device is constructed from a lattice-like network of flexible rods and spars, providing a three dimensional weave-like body for the pressure member. The pitch of the lattice may be such that additional aeration is allowed for via the lattice, whilst also providing sufficient separation of skin folds in use.

The dimensions of the device may vary depending on application and the physical size of the intended user. For example, in an embodiment, a “medium” sized device for haemorrhoids may have approximate length L twice that of the width W, and a depth D or thickness roughly half of the width.

The aperture may be round, or oblong or square, and should be sized so as provide aeration to the wound whilst maintaining resilience when the pressure member is placed so as to engage, conform and grip the skin folds. For example, the aperture may be about one quarter to half of the length L, and about a quarter of the width W across.

It will be apparent to those skilled in the art that the actual dimensions and shape chosen depend on the application. For example, in an embodiment for separating abdominal skin folds of fat on an obese person, the device may be much larger in all three dimensions, with more than one aperture to provide aeration.

However, it will be readily apparent to those skilled in the art that the device may be useful in any area where skin folds or overlaps or creases, and hence covers or obscures a wound therein. The device may hence also be used in other areas of the body where wounds such as a sore or blister or fistula or abraded flesh occur, and where aeration of the wound may provide comfort and aid healing. Areas include, but are not limited to the anal or perineal region and the abdominal region where “skin rolls” on overweight or obese bodies can be problematic and cause sores and blisters therebetween.

In another aspect the present invention provides a method for providing relief to covered wounds, the method comprising compressing a medical device as defined above, placing the compressed medical device between skin folds in the vicinity of the covered wound, and releasing the medical device to separate and hold apart the skin folds to provide aeration of the wound.

In the above, a medical device for covered wound relief is described. The device comprises a substantially annular pressure member with at least one aperture therethrough. It will be apparent to those skilled in the art that various forms and sizes of the substantially annular pressure member, beyond that of a simple ring, are possible depending on the intended wound application and whether optional conformance with the skin folds is desired. The device is preferably resilient and remains in-situ once placed and hence provides aeration to an otherwise covered wound.

In an embodiment the skin folds are preferably buttocks, and the wound is a haemorrhoid, a fistula or an anal fissure.

In one embodiment, there is provided a medical device for covered wound relief, comprising a pressure member for separating a skin fold covering or overlapping a wound, and wherein the pressure member has at least one aperture extending therethrough for aeration of the wound, and an internal channel extending around the aperture.

In one embodiment, the channel is an enclosed internal channel.

In one embodiment, the channel is an embedded channel.

In one embodiment, the channel contains a medium.

In one embodiment, the medium comprises a fluid, optionally a gel.

In one embodiment, the medium is heatable and/or coolable.

In one embodiment, the medium comprises cold pack fluid or gel.

In one embodiment, the channel comprises a first section, a second section, and a breakable membrane separating the first section from the second section, the membrane being breakable by flexing or compressing the device.

In one embodiment, the first section comprises a first medium and the second section comprises a second medium.

In one embodiment, the first and second mediums cause an endothermic reaction when mixed.

In one embodiment, the first chemical and second medium cause an exothermic reaction when mixed.

In one embodiment, the skin folds are in the perineal or anal region.

In one embodiment, the skin folds are buttocks.

In one embodiment, the skin folds are formed in the abdominal region.

In one embodiment, the pressure member is formed of a resilient material.

In one embodiment, the pressure member is an annular pressure member.

In one embodiment, the pressure member is ovoidal in form.

In one embodiment, the pressure member is rhomboidal in form.

In one embodiment, the pressure member comprises an inner surface defining the at least one aperture and an outer surface defining an outer periphery of the pressure member.

In one embodiment, the outer surface includes first and second surface sections which extend radially outwardly in spaced relation to the inner surface.

In one embodiment, one surface section has an arcuate form.

In one embodiment, the surface sections both have an arcuate form.

In one embodiment, the surface sections are convex surfaces.

In one embodiment, the surface sections are concave surfaces.

In one embodiment, each arcuate surface is symmetric.

In one embodiment, each arcuate surface is asymmetric.

In one embodiment, the surface sections comprise a first major element which extends substantially in a first plane, and a second, minor element which extends substantially in a second plane which encloses an obtuse angle with the first plane.

In one embodiment, the outer surface comprises an outer sidewall which extends between the radially outer periphery of the first and second surface sections.

In one embodiment, the outer sidewall is inclined relative to an axial direction extending between the first and second surface sections.

In one embodiment, there is provided a medical device for covered wound relief, comprising an annular pressure member for separating a skin fold covering or overlapping a wound, and wherein the pressure member has at least one aperture extending therethrough, and wherein the pressure member comprises an inner surface defining the at least one aperture and an outer surface defining the periphery of the pressure member, and wherein the outer surface includes first and second surface sections which extend radially outwardly in spaced relation to the inner surface, and wherein the outer surface comprises an outer sidewall which extends between the radially outer periphery of the first and second surface sections, and wherein the outer sidewall is inclined relative to an axial direction extending between the first and second surface sections.

In one embodiment, one surface section has an arcuate form.

In one embodiment, the surface sections both have an arcuate form.

In one embodiment, the surface sections are convex surfaces.

In one embodiment, the surface sections are concave surfaces.

In one embodiment, each arcuate surface is symmetric.

In one embodiment, each arcuate surface is asymmetric.

In one embodiment, the sidewall is inclined of from about 5 degrees to about 30 degrees relative to an axial direction extending between the first and second surface sections.

In one embodiment, the pressure member is formed from silicon, natural or synthetic rubber, foam or plastic.

In one embodiment, the outer surface of the pressure member is textured.

In one embodiment, the at least one aperture is provided with a micromesh covering.

In one embodiment, the pressure member comprises more than one aperture.

In one embodiment, the pressure member has an outer surface portion and an inner portion, and wherein the outer surface portion is absorbent or hydrophilic.

In one embodiment, the outer surface portion is impregnated with one or more antiseptic, antibacterial or sterilizing compositions.

In one embodiment, the pressure member is formed from a lattice-like network of resilient material.

In one embodiment, there is provided a method for providing relief to covered wounds, comprising: compressing a medical device according to any embodiment, placing the compressed medical device between skin folds in the vicinity of the covered wound, releasing the medical device to separate and hold apart the skin folds to provide aeration of the wound.

In one embodiment, the skin folds are buttocks and the wound is a haemorrhoid, a fistula, a seton, an anal fissure or Pylonidial cysts.

In one embodiment, there is provided a device substantially as hereinbefore described with reference to the accompanying drawings.

Further optional features will be apparent from the following description and accompanying claims.

Embodiments of the present invention will now be described, by way of example only with reference to the accompanying drawings, in which:

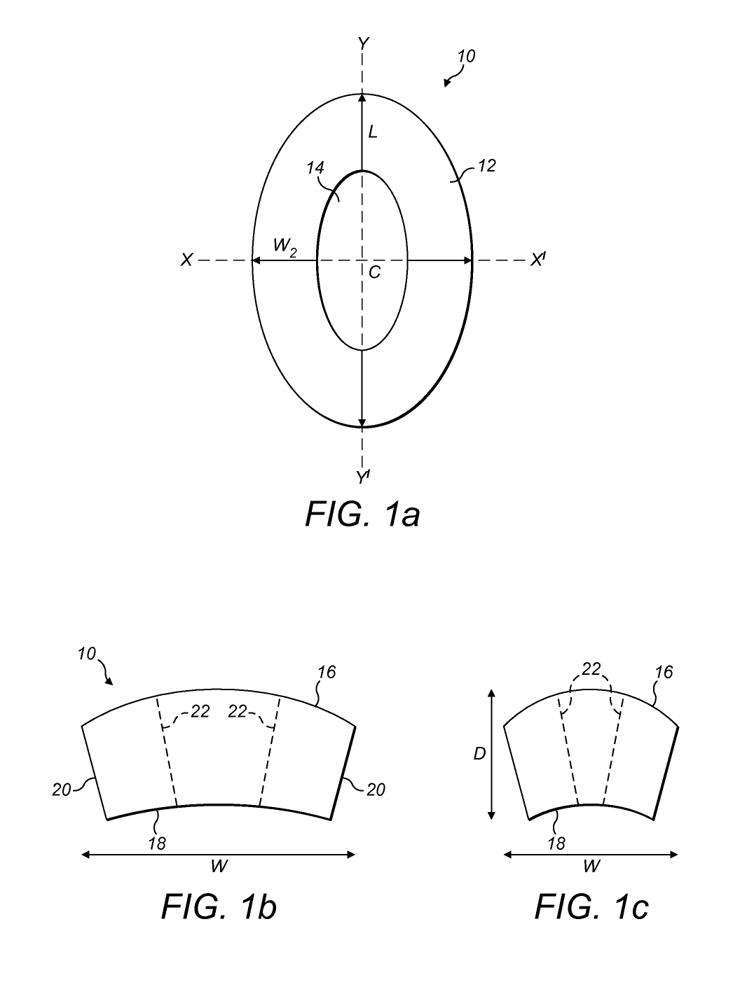

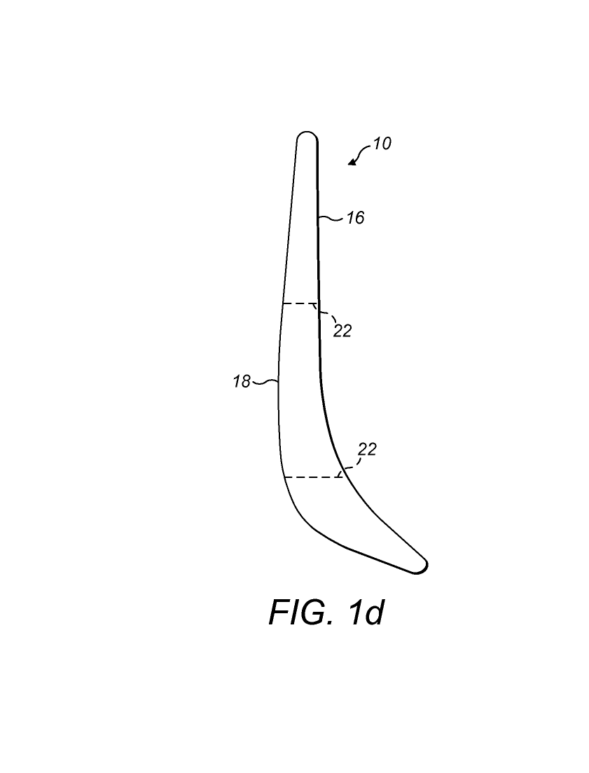

FIG. 1a, FIG. 1b and FIG. 1c is show a device according to an embodiment of the invention in plan, side and end views, respectively; FIG. 1d shows an alternative side view;

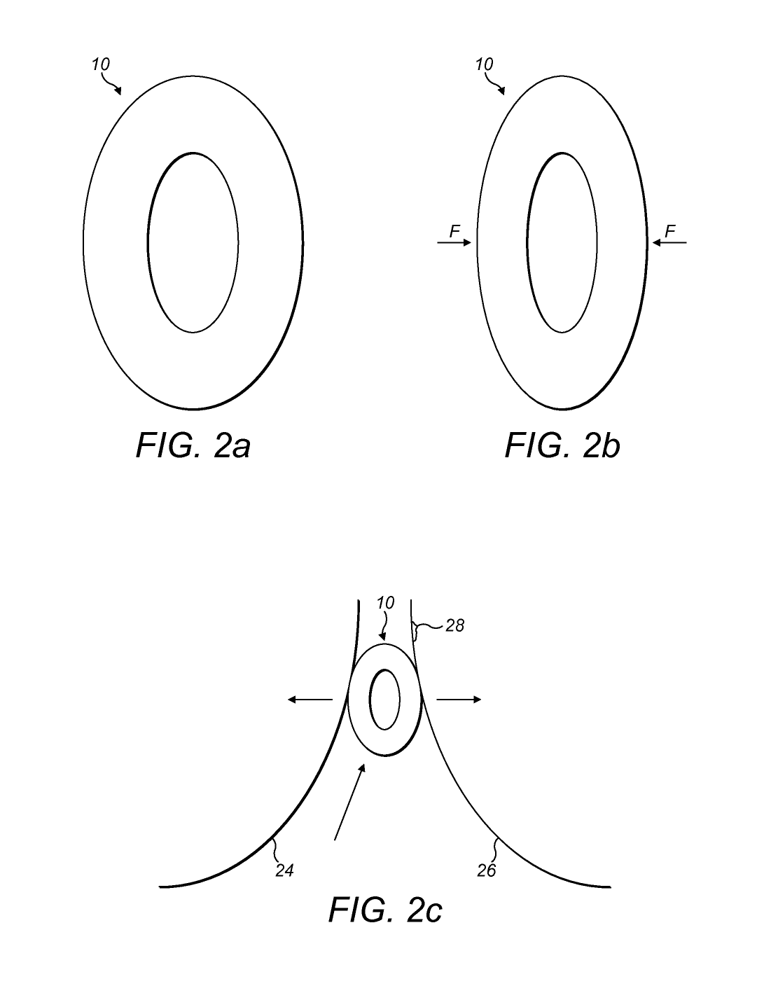

FIG. 2a and FIG. 2b and FIG. 2c illustrate the device of FIG. 1 in an uncompressed state, a compressed state, and placement of the device between the buttocks of a person in the perineal or anus region respectively;

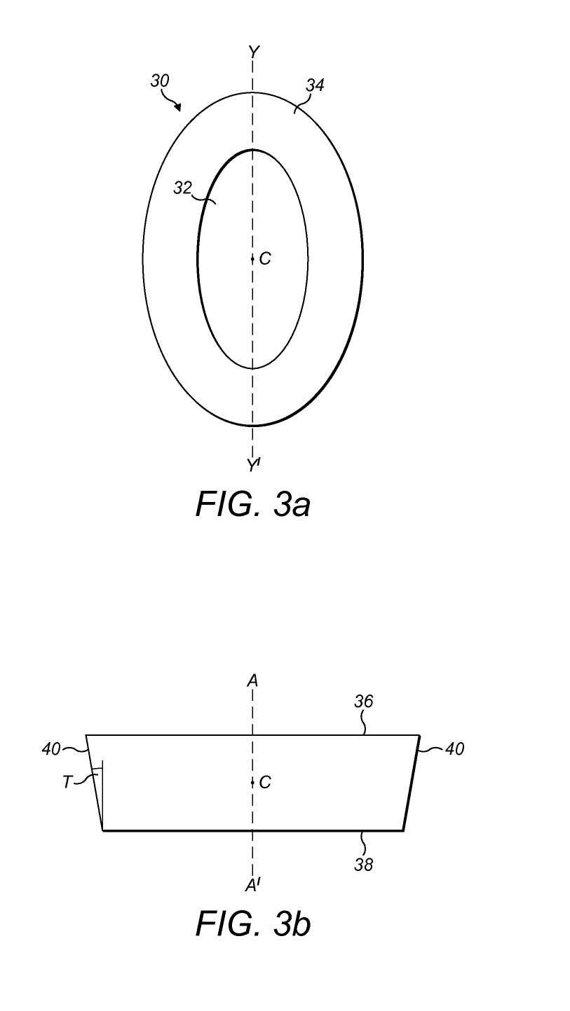

FIG. 3a and FIG. 3b show a device according to another embodiment of the invention in plan and side views respectively;

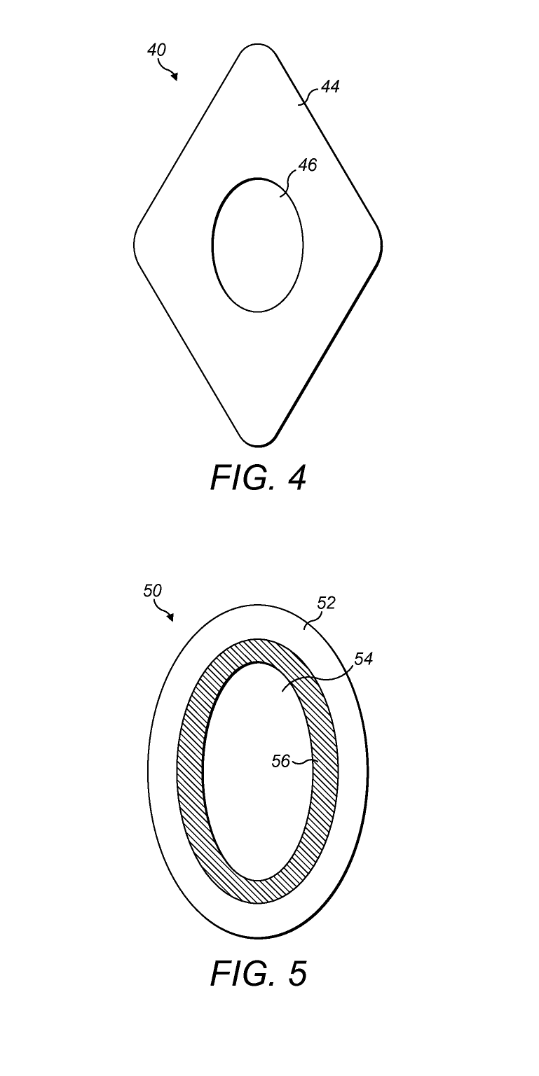

FIG. 4 illustrates an embodiment of the invention in which the device has a rhomboid form;

FIG. 5 is a plan view of yet another embodiment of the device having a channel according to the invention;

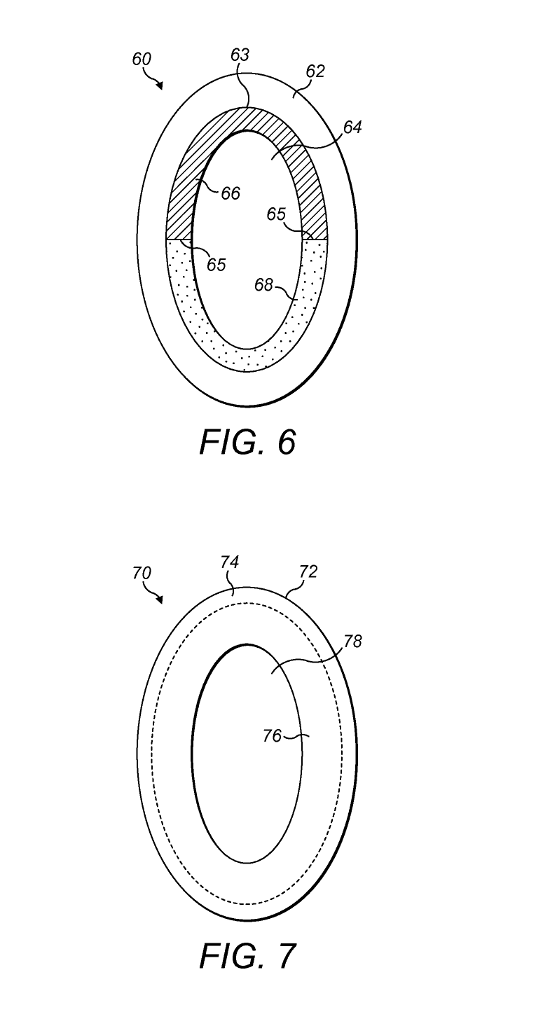

FIG. 6 shows a plan view of another embodiment of the device having a channel according to the invention;

FIG. 7 is a plan view of yet another embodiment of the device according to the invention;

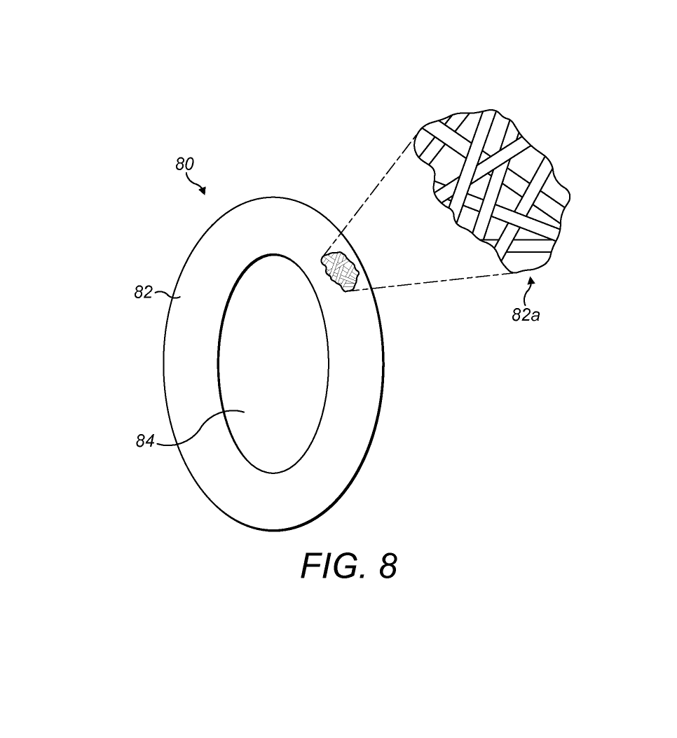

FIG. 8 is a schematic plan view of an embodiment of the device having a lattice-like pressure member arrangement according to the invention.

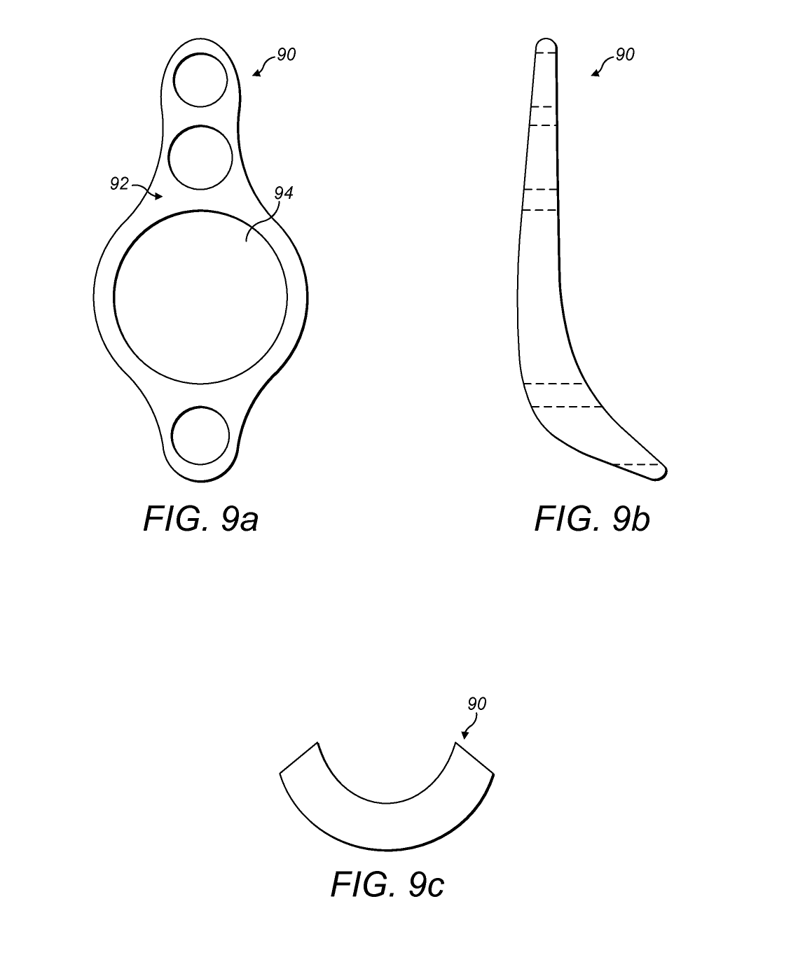

FIG. 9a, FIG. 9b and FIG. 9c show a device according to another embodiment of the invention in plan, side and end views, respectively;

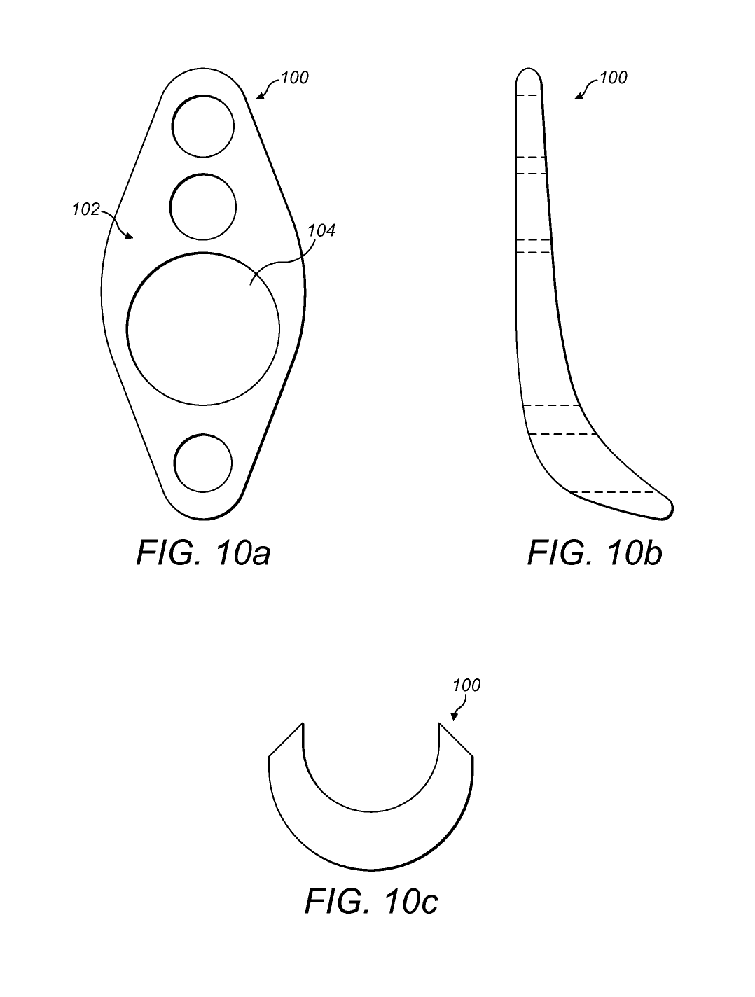

FIG. 10a, FIG. 10b and FIG. 10c show a device according to yet another embodiment of the invention in plan, side and end views, respectively.

FIGS. 1a, 1b and 1c illustrate an embodiment of a medical device in plan, side and end views, respectively.

In FIG. 1a, the medical device 10 comprises an annular pressure member 12. In this embodiment, the pressure member 12 has a generally annular oval or ellipse like shape, with a centrally located aperture 14 with a centre C. The aperture extends completely through the body of the pressure member 12. The pressure member 12 has a length L along the long (major) axis Y-Y′, and a width W along the short (minor) axis.

The pressure member 12 can be made preferably from any sufficiently resilient and flexible material such as natural or synthetic rubber, low, medium or high density foam, silicone, plastic or a mixture of these.

FIG. 1b shows a side view of the same device along the Y-Y′ axis. In this embodiment the pressure member has an upper surface 16 and a lower surface 18 separated by a tapered sidewall 20. The aperture 14 is defined by an inner surface or wall 22.

FIG. 1c shows an end view of the same device along the X-X′ axis. The pressure member 12 has an approximate depth D as shown in the Figure.

In this embodiment the upper surface 16 and lower surface 18 form curved surfaces and are convex with respect to each other as shown in FIG. 1b and FIG. 1c.

In another embodiment, the upper surface 16 and lower surface 18 are arcuate and asymmetric as shown in FIG. 1d.

In one example, the length L is about 8 cm, the width W is about 3-4 cm, and the depth D is roughly 2 cm. The aperture is about one quarter to half of the length L, in this example 2-3.5 cm, and about a quarter of the width W across, in this example 1 cm.

As can be seen from FIGS. 1a and 1b, the device in this embodiment is roughly “wedge” shaped to ease application of the device, which will now be described with reference to FIG. 2.

FIG. 2a is a plan view of an embodiment of the device in its natural or decompressed state. In use, the user compresses or squeezes the device by applying a force F with their fingers as illustrated in FIG. 2b. The user then places or inserts the device between skin folds 24, 26 in the vicinity of a wound 28. In this example, as shown in FIG. 2c, the skin folds are buttock cheeks or buttocks and the wound is a haemorrhoid. Other examples of wounds include anal fissures, fistulas, sores and blisters and the like.

As shown in FIG. 2c, the device is inserted close to the wound by the user who then releases the device. The device returns to its natural form or state and hence parts or separates the skin folds conforming to it. The aperture then allows increased aeration of the wound.

In this embodiment the preferably tapered and curved profile of the device aids easier insertion or placement of the device with the walls conforming to and separating the skin folds.

In another embodiment as shown in FIG. 3a and FIG. 3b, the device 30 has a pressure member 34 that is substantially oval in plan section with an aperture 32 roughly centrally located. The device has an outer surface comprising an upper surface 36 and a lower surface 38 that are essentially flat with respect to each other and orthogonal with respect to a vertical axis A-A′ passing through the centre of the device as depicted in FIG. 3b.

The device has sidewalls 40 extending around the periphery of the device 30 between the upper surface 36 and the lower surface 38.

In this embodiment the sidewalls are tapered inwards from the upper to the lower surface at an angle T with respect to the vertical axis or normal A-A′ as shown in FIG. 3b.

In another embodiment the device 42 has an annular pressure member 44 that is approximately rhomboid in form with an aperture 46 passing through the body of the pressure member 44 as shown in FIG. 4.

In another embodiment the device 10 may be provided with a pressure member 12 that is substantially toroidal (doughnut like) in form.

In another embodiment as illustrated in FIG. 5, the device 50 has a pressure member 52 with an aperture 54 for providing aeration. The pressure member 52 has an internal embedded channel 56 extending around the aperture 54.

In an embodiment, the channel 56 is a waterproof sealed tube or cavity embedded in the foam body, and extending around the aperture.

In an embodiment the channel 56 contains a cold pack cooling fluid, such as the commercially available reusable “blue gel” cold pack fluids which may be a mixture of water, propylene glycol and cellulose such as sodium carboxymethyl cellulose or hydroxyethyl cellulose, or vinyl coated silica gel.

Those skilled in the art will appreciate that other suitable chemicals may be employed.

In use, the user places the device 50 in a cooler or freezer for some time prior to applying the device 50. Hence a cooling effect in addition to aeration is provided which aids comfort and relief from itching.

In another embodiment, the embedded channel 56 surrounds the aperture 54 and extends close to the outer surface or periphery of the device to aid thermal transfer from the device to the skin fold area in which it is intended for use.

In another embodiment as depicted in FIG. 6, the device 60 has a pressure member 62 with an aperture 64. The pressure member is provided with an internal embedded channel 63 extending around the aperture 64. The channel is provided with a breakable membrane 65 which separates the channel into a first section 66 and a second section 68. The first section comprises a first composition and the second section comprises a second composition which when mixed cause an endothermic reaction.

Suitable first compositions comprise chemicals such as ammonium salts, for example, ammonium nitrate. The second composition may comprise distilled water.

The breakable membrane 65 separating the first composition from the second composition is broken with the aid of the user by flexing or compressing the device prior to placement as previously described with reference to FIG. 2b. The compositions then mix and an endothermic reaction takes place which cools the device down providing relief as described before.

In another embodiment as shown in FIG. 7 the device 70 has a pressure member 72 and an aperture 78. The device has an outer surface portion 74 and an inner portion 76. The outer surface portion 74 is absorbent relative to the inner portion to enable antibacterial, antibiotic, antiseptic or sterilizing agents to be applied to the device prior to use.

In an embodiment, the pressure member 72 may be formed from high density foam 76 with an outer surface portion 74 of low density form. This may be achieved using modern injection moulding or extruding techniques.

In another embodiment, the outer surface portion 74 may comprise surgical tissue that is adhered to the contours of the pressure member during manufacture.

In another embodiment, the pressure member 12, 34, 44, 52, 62, 72, 82 may be roughened or textured to enhance grip and engagement with skin folds.

In another embodiment the medical device 80 has a pressure member 82 with an aperture 84 as shown in FIG. 8. In this embodiment the pressure member 82 is constructed from a lattice-like network of flexible rods and spars 82a, providing a three dimensional weave-like body or “birds nest” for the pressure member. The pitch of the lattice may be chosen so that additional aeration is allowed for via the lattice, whilst also providing sufficient separation of skin folds in use. In this embodiment, the pressure member may be formed from synthetic rubber or silicone.

FIGS. 9a, 9b and 9c illustrate an embodiment of a medical device in plan, side and end views, respectively.

In FIG. 9a, the medical device 90 comprises a pressure member 92. In this embodiment, the pressure member has an an ovoidal shape, with four apertures. The apertures extend completely throughout the body of pressure member 92.

FIG. 9b shows a side view of the same device.

FIGS. 10a, 10b and 10c illustrate an embodiment of a medical device in plan, side and end views, respectively.

In FIG. 10a, the medical device 100 comprises a pressure member 102. In this embodiment, the pressure member has an an ovoidal shape, with four apertures. The apertures extend completely throughout the body of pressure member 102.

FIG. 10b shows a side view of the same device.

Finally, it will be understood that the present invention has been described in its preferred embodiments and can be modified in many different ways without departing from the scope of the invention as defined by the appended claims.

1. A skin fold separating device for covered wound relief, comprising:
an annular pressure member for separating a skin fold covering or overlapping a wound, and
wherein the pressure member has at least one aperture extending therethrough, and
wherein the pressure member comprises an inner surface defining the at least one aperture and an outer surface defining the periphery of the pressure member, and
wherein the outer surface includes first and second surface sections which extend radially outwardly in spaced relation to the inner surface, and
wherein the outer surface comprises an outer sidewall which extends between the radially outer periphery of the first and second surface sections, and
wherein the outer sidewall is inclined relative to an axial direction extending between the first and second surface sections.

2. The device of claim 1, further comprising an internal channel extending around the aperture, optionally wherein the channel is an enclosed internal channel.

3. The device according to claim 2, wherein the channel contains a medium, optionally the medium comprises a fluid and/or gel.

4. The device according to claim 3, wherein the medium is heatable and/or coolable.

3. The device according to claim 3, wherein the medium comprises cold pack fluid or gel.

4. The device according to claim 2, wherein the channel comprises a first section, a second section, and a breakable membrane separating the first section from the second section, the membrane being breakable by flexing or compressing the device.

5. The device of claim 4, wherein the first section comprises a first medium and the second section comprises a second medium.

6. The device of claim 5, wherein the first and second mediums cause an endothermic reaction when mixed or an exothermic reaction when mixed.

7. The device according to claim 1, wherein the skin folds are in the perineal, anal region, buttocks or abdominal region.

8. The device according to claim 1, wherein the pressure member is formed of a resilient material, silicon, natural or synthetic rubber, foam or plastic, optionally the pressure member is formed of a lattice-like network of resilient material.

9. The device according to claim 1, wherein one surface section has an arcuate form, or the surface sections both have an arcuate form.

10. The device according to claim 9, wherein the surface sections are convex surfaces or concave surfaces.

11. The device of according to claim 9, wherein each arcuate surface is symmetric or asymmetric.

12. The device according to claim 1, wherein the sidewall is inclined of from about 5 degrees to about 30 degrees relative to an axial direction extending between the first and second surface sections.

13. The device of according to claim 1, wherein the outer surface of the pressure member is textured.

14. The device according to claim 1, wherein the at least one aperture is provided with a micromesh covering.

15. The device according to claim 1, where the pressure member comprises more than one aperture.

16. The device of according to claim 1, wherein the pressure member has an outer surface portion and an inner portion, and wherein the outer surface portion is absorbent or hydrophilic, optionally wherein the outer surface portion is impregnated with one or more antiseptic, antibacterial or sterilizing compositions.

17. A method for providing relief to covered wounds, comprising:
providing a skin fold separating device for covered wound relief, comprising an annular pressure member for separating a skin fold covering or overlapping a wound, and wherein the pressure member has at least one aperture extending therethrough, and wherein the pressure member comprises an inner surface defining the at least one aperture and an outer surface defining the periphery of the pressure member, and wherein the outer surface includes first and second surface sections which extend radially outwardly in spaced relation to the inner surface, and wherein the outer surface comprises an outer sidewall which extends between the radially outer periphery of the first and second surface sections, and wherein the outer sidewall is inclined relative to an axial direction extending between the first and second surface sections compressing the medical device,
placing the compressed medical device between skin folds in the vicinity of the covered wound,
releasing the medical device to separate and hold apart the skin folds to provide aeration of the wound.

18. A method according to claim 17, wherein the skin folds are buttocks and the wound is a haemorrhoid, a fistula, a seton, an anal fissure or Pylonidial cysts.
